(19) United States
(12) Patent Application Publication
(10) Pub. No.: US 2022/0008499 A1
(43) Pub. Date: Jan. 13, 2022

(54) COMPOUNDS, COMPOSITIONS AND METHODS FOR TREATING SEPSIS

(71) Applicant: FERRING B.V., Hoofddorp (NL)

(72) Inventors: Anne Louise Søberg KJØLBYE, Vedbaek (DK); Nis Agerlin WINDELØV, Vanløse (DK); Derek C. ANGUS, Pittsburgh, PA (US); James A. RUSSELL, Vancouver (CA)

(73) Assignee: FERRING B.V., Hoofddorp (NL)

(21) Appl. No.: 17/294,169

(22) PCT Filed: Nov. 14, 2019
(86) PCT No.: PCT/IB2019/059788

§ 371 Date: May 14, 2021

Related U.S. Application Data
(60) Provisional application No. 62/767,889, filed on Nov. 15, 2018.

(51) Int. Cl. A61K 38/095; A61K 45/06; A61P 37/02; A61K 9/00

(52) U.S. Cl. CPC A61K 38/095; A61K 9/0019; A61P 37/02; A61K 45/06

(57) ABSTRACT

There are disclosed herein compositions comprising selepressin for use in treating sepsis in a patient. The composition comprising selepressin may be administered within six hours from when the patient requires vasopressor therapy. The patients may have a serum lactate concentration of less than about 2 mmol/L prior to treatment and/or a ratio of partial pressure arterial oxygen to fraction of inspired oxygen (PaO₂/FᵢO₂) of greater than about 200 prior to treatment. Related methods also are disclosed.

### CROSS-REFERENCE TO RELATED PATENT APPLICATIONS

This application claims priority to U.S. provisional application 62/767,889, filed Nov. 15, 2018 the entire contents of which is incorporated herein by reference.

### FIELD

Described herein are compounds, compositions, and methods for treating sepsis.

### BACKGROUND

Sepsis is a syndrome of physiologic, pathologic, and biochemical abnormalities induced by a dysregulated host response to infection leading to organ dysfunction. Typically, the infection is bacterial, although sepsis can be triggered by fungal, viral or parasitic infection.

Sepsis may lead to vasodilatation and increased capillary permeability leading to hypotension and tissue hypoxia. In more severe cases, patients with sepsis may develop need for vasopressor support despite adequate fluid resuscitation, in which case there is a high risk of multiple organ failure, protracted intensive care, and death.

Vasopressors are agents that raise blood pressure. Vasopressors are also known as antihypotensive agents, vasopressor agents or pressors.

Septic shock denotes the subpopulation of patients with sepsis who require vasopressor treatment and who have a serum lactate concentration of ≥2 mmol/L. Treatment of sepsis and septic shock remains a substantial unmet medical need. Norepinephrine has traditionally been the vasopressor of choice in the treatment of sepsis, recommended as the first-line vasopressor in the Surviving Sepsis Guidelines¹. However, vasopressin infusion has been used to replace norepinephrine to maintain adequate systemic arterial pressure (e.g., in patients refractory to norepinephrine). In a large, multicenter, randomized, double-blind, norepinephrine-controlled trial (the Vasopressin and Septic Shock Trial [VASST]), vasopressin was associated with decreased mortality compared with norepinephrine in patients requiring 5-14 ug/min of norepinephrine at enrollment, although the overall mortality was not different. Vasopressin is an endogenous ligand for three subtypes of vasopressin receptor (V1a, V1b, and V2), and it also activates the oxytocin receptor.

Owing to the prevalence and seriousness of sepsis and septic shock, there is an ongoing need for new uses of compounds and new methods for treating sepsis and septic shock.

### SUMMARY OF THE INVENTION

Provided herein are compositions comprising selepressin for use in treating sepsis in a patient requiring vasopressor therapy, wherein the composition comprising selepressin is administered to the patient within six hours from when the patient requires the vasopressor therapy. In some aspects, the patient has, or is identified as having, a ratio of partial pressure arterial oxygen to fraction of inspired oxygen (PaO₂/FiO₂) of greater than about 200 prior to treatment. Additionally or alternatively, in some aspects, the patient has, or is identified as having, a serum lactate concentration of less than about 2 mmol/L prior to treatment, or has a serum lactate concentration of less than 2 mmol/L prior to treatment or is identified as having a serum lactate concentration of less than about 2 mmol/L prior to treatment.

Also provided are compositions comprising selepressin for use in treating sepsis in a patient, wherein the patient has a ratio of partial pressure arterial oxygen to fraction of inspired oxygen (PaO₂/FiO₂) of greater than about 260 prior to treatment. In some aspects, the patient is identified as having a ratio of partial pressure arterial oxygen to fraction of inspired oxygen (PaO₂/FiO₂) of greater than about 260 prior to treatment, or greater than about 300 prior to treatment.

Also provided are methods of treating sepsis in a patient requiring vasopressor therapy, comprising administering a composition comprising selepressin to the patient within six hours from when the patient requires the vasopressor therapy In some aspects, the patient has, or is identified as having, a serum lactate concentration of less than about 2 mmol/L prior to treatment, or has a serum lactate concentration of less than 2 mmol/L prior to treatment or is identified as having a serum lactate concentration of less than about 2 mmol/L prior to treatment. Additionally or alternatively, in some aspects, the patient has, or is identified as having, a ratio of partial pressure arterial oxygen to fraction of inspired oxygen (PaO₂/FiO₂) of greater than about 200 prior to treatment, greater than about 260 prior to treatment, or greater than about 300 prior to treatment.

Also provided are methods of treating sepsis comprising administering a composition comprising selepressin to a patient in need thereof, wherein the patient has a serum lactate concentration of less than 2 mmol/L prior to treatment, or is identified as having a serum lactate concentration of less than about 2 mmol/L prior to treatment.

Also provided are methods of treating sepsis comprising administering a composition comprising selepressin to a patient in need thereof, wherein the patient has a ratio of partial pressure arterial oxygen to fraction of inspired oxygen (PaO₂/FiO₂) of greater than about 200 prior to treatment, greater than about 260, or greater than about 300, prior to treatment, or is identified as having a ratio of partial pressure arterial oxygen to fraction of inspired oxygen (PaO₂/FiO₂) of greater than about 200, greater than about 260, or greater than about 300, prior to treatment.

In any aspects, of the compositions and methods described herein, the sepsis may be vasopressor dependent sepsis. In any aspects of the compositions and methods described herein, the sepsis may be septic shock.

In some aspects of the compositions and methods described herein, the selepressin is administered by continuous intravenous infusion at a starting intravenous infusion dose rate between about 1.7 ng/kg/min and about 5.0 ng/kg/min; and a maximum intravenous infusion dose rate between about 2.5 ng/kg/min and about 7.5 ng/kg/min.

In any aspects of the compositions and methods described herein, the selepressin may be administered intravenously or subcutaneously. In any aspects of the compositions and methods described herein, the selepressin may be administered by continuous intravenous infusion at a dose rate from about 1.7 ng/kg/min to about 7.5 ng/kg/min. In any aspects of the compositions and methods described herein, the selepressin may be administered together with a further antihypotensive agent. In any aspects of the compositions and methods described herein, the selepressin may be administered together with an inotropic agent.

### BRIEF DESCRIPTION OF THE DRAWINGS

FIG. 1 shows a forest plot by baseline PaO₂/FiO₂ subgroups for vasopressor free days up to day 30 of subjects treated as described in the examples.

### DETAILED DESCRIPTION

Described herein is selepressin or a composition comprising selepressin for use in treating sepsis, and methods of treating sepsis comprising administering selepressin.

Selepressin is a cyclic nonapeptide vasopressin analog with high affinity and selectivity for the human vasopressin V1a receptor relative to the V1b, V2, and oxytocin receptors and with no known affinity for other receptors, ion channels, or transporters³. Selepressin has the following formula as set out in Formula 1a (peptide formula) and 1b (skeletal formula):

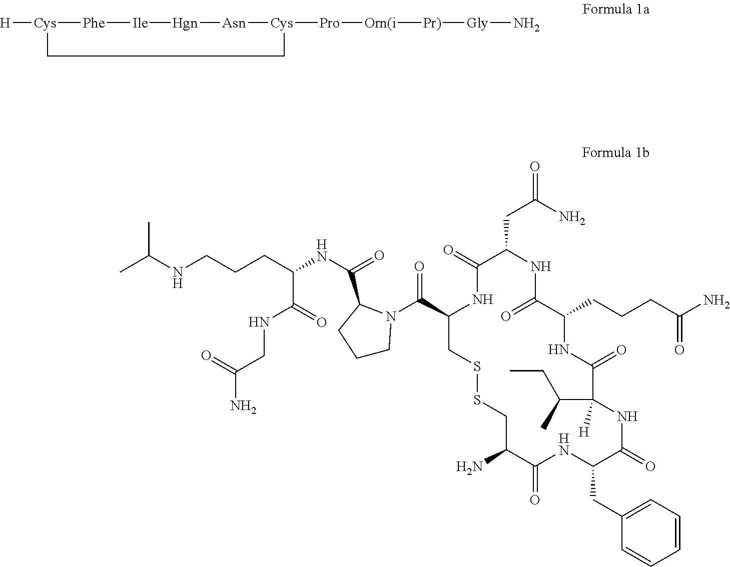

According to an aspect of the invention there is provided selepressin or a composition comprising selepressin for use in treating sepsis in a patient requiring vasopressor therapy, wherein the selepressin or composition comprising selepressin is administered to the patient within six hours from when the patient requires the vasopressor therapy. There is also provided a method of treating sepsis in a patient requiring vasopressor therapy, comprising administering selepressin or a composition comprising selepressin to the patient within six hours from when the patient requires vasopressor therapy. There is also provided selepressin or a composition comprising selepressin for use in the manufacture of a medicament for the treatment of sepsis in a patient requiring vasopressor therapy, wherein the treatment comprises administering the selepressin or the composition comprising selepressin to the patient within six hours from when the patient requires the vasopressor therapy. The applicant has found that administering selepressin within six hours from when the patient requires vasopressor therapy leads to improved mortality and improved patient outcomes, for example as measured by pressor and mechanical ventilator-free days (P&VFDs).

Vaseopressor therapy includes the administration of a vasopressor. Patients having sepsis may require vasopressor therapy if the patient does not respond to fluid resuscitation, for example, if the patient does not respond to the administration of about 30 ml/kg of intraveneous fluid (or another volume as deemed needed to correct potential intravascular hypovolemia) and the patient has a mean arterial blood pressure below about 65 mmHg.

The selepressin or the composition comprising selepressin may be administered within six hours from when the patient does not respond to fluid resuscitation. For example, the selepressin or the composition comprising selepressin may be administered within six hours from when the patient does not respond to the administration of about 30 mL/kg of intravenous fluid (or another volume as deemed needed to correct potential intravascular hypovolemia) and the patient has a mean arterial blood pressure below about 65 mmHg.

The sepsis may be septic shock or vasopressor dependent sepsis.

The patient may have a serum lactate concentration of less than 2 mmol/L prior to treatment.

Herein, the term “prior to treatment” means prior to administration of the selepressin or the composition comprising selepressin.

The patient may be identified (for example prior to treatment) as having a serum lactate concentration of less than about 2 mmol/L prior to treatment. The patient may, for example, be selected for treatment based on having a serum lactate concentration of less than about 2 mmol/L prior to treatment.

The applicant has found that patients who have a serum lactate concentration of less than about 2 mmol/L prior to treatment exhibit improved mortality and improved patient outcomes, for example as measured by pressor and mechanical ventilator-free days (P&VFDs).

The patient may have a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may be identified (for example prior to treatment) as having a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may for example be selected for treatment based on having a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment.

The applicant has found that patients who have a ratio of partial pressure arterial oxygen to fraction of inspired oxygen (PaO₂/FiO₂) greater than about 200 prior to treatment exhibit improved mortality and improved patient outcomes, for example as measured by pressor and mechanical ventilator free days.

The selepressin or the composition comprising selepressin may be administered to the patient intravenously, intraperitoneally, intramuscularly, nasally or subcutaneously. Typically, the selepressin or composition comprising selepressin is administered intravenously or subcutaneously. The selepressin or composition comprising selepressin may be administered by infusion, for example continuous infusion, for example an intravenous or subcutaneous infusion, for example intravenous or subcutaneous continuous infusion. Typically, administration is by an intravenous continuous infusion.

The composition comprising selepressin may be in the form of powders, microparticles, granules, suspensions or solutions, formulated for the intended route of administration.

The composition may comprise for example at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3rd Ed., American Pharmaceutical Association, USA and Pharmaceutical Press UK, 2000.

The composition may be formulated for intravenous administration, for example intravenous continuous infusion. Accordingly, the composition may comprise a sterile aqueous preparation of the selepressin, preferably isotonic with the blood of the recipient. This aqueous preparation may be formulated according to known methods using suitable dispersing or wetting agents and suspending agents. The preparation may also be a sterile injectable solution or suspension in a diluent or solvent, for example a solution in 1,3-butane diol. Other acceptable diluents may include water, Ringer's solution, and isotonic sodium chloride solution. Sterile, fixed oils may be employed as a solvent or suspending medium. Bland fixed oils, including synthetic mono or di-glycerides, and fatty acids, such as oleic acid, may also be used.

The selepressin may be administered in amounts up to about 7.5 nanogram/kilogram/minute (ng/kg/min), for example between about 1.25 ng/kg/min and about 7.5 ng/kg/min, for example between about 1.25 ng/kg/min and about 1.7 ng/kg/min or between about 1.7 ng/kg/min and about 2.5 ng/kg/min, or between about 2.5 ng/kg/min and about 3.75 ng/kg/min, or between about 3.5 ng/kg/min and about 5.25 ng/kg/min, or between about 5.0 ng/kg/min and about 7.5 ng/kg/min.

The selepressin may be administered at an initial infusion dose rate which increases to a maximum infusion dose rate over time. The initial infusion dose rate may be two-thirds of the maximum infusion dose rate. The initial infusion dose rate may be between about 1.7 ng/kg/min and about 5.0 ng/kg/min, for example, about 1.7 ng/kg/min, about 2.5 ng/kg/min, about 3.5 ng/kg/min or about 5 ng/kg/min. The maximum infusion dose rate may be between about 2.5 ng/kg/min and about 7.5 ng/kg/min, for example, about 2.5 ng/kg/min, about 3.75 ng/kg/min, about 5.25 ng/kg/min or about 7.5 ng/kg/min.

The infusion dose rate may start at an initial infusion dose rate of about 1.7 ng/kg/min and increase to a maximum infusion dose rate of about 2.5 ng/kg/min; or the infusion dose rate may start at an initial infusion dose rate of about 2.5 ng/kg/min and increase to a maximum infusion dose rate of about 3.75 ng/kg/min; or the infusion dose rate may start at an initial infusion dose rate of about 3.5 ng/kg/min and increase to a maximum infusion dose rate of about 5.25 ng/kg/min; or the infusion dose rate may start at an initial infusion dose rate of about 5.0 ng/kg/min and increase to a maximum infusion dose rate of about 7.5 ng/kg/min. The above mentioned dose rates may be for an intravenous or subcutaneous infusion, for example intravenous or subcutaneous continuous infusion.

The treatment duration may be until recovery from the need of vasopressor treatment. If the need of vasopressor treatment subsides and recurs, the treatment can be restarted.

The selepressin or composition comprising selepressin may be administered together with a further antihypotensive agent (vasopressor), for example a catecholamine, for example norepinephrine.

The selepressin or composition comprising selepressin may be administered together with an inotropic agent, for example milrinone, dobutamine, and/or levosimendan.

According to another aspect of the invention there is provided selepressin or a composition comprising selepressin for use in treating sepsis in a patient, wherein the patient has a serum lactate concentration of less than about 2 mmol/L prior to treatment. There is also provided a method of treating sepsis in a patient, comprising administering selepressin or a composition comprising selepressin to the patient, wherein the patient has a serum lactate concentration of less than about 2 mmol/L prior to treatment. There is also provided a use of selepressin or a composition comprising selepressin in the manufacture of a medicament for the treatment of sepsis in a patient, wherein the patient has a serum lactate concentration of less than about 2 mmol/L prior to treatment.

Herein, the term “prior to treatment” means prior to administration of the selepressin or the composition comprising selepressin.

For example, the patient may be identified (for example prior to treatment) as having a serum lactate concentration of less than about 2 mmol/L prior to treatment. For example, the patient may be selected for treatment based on having a serum lactate concentration of less than about 2 mmol/L prior to treatment.

The applicant has found that patients who have a serum lactate concentration of less than about 2 mmol/L prior to treatment exhibit improved mortality and improved patient outcomes, for example as measured by pressor and mechanical ventilator free days.

The sepsis may be vasopressor dependent sepsis.

The selepressin or the composition comprising selepressin may be administered within six hours from when the patient requires vasopressor therapy.

Patients having sepsis may require vasopressor therapy, for example the administration of a vasopressor, if the patient does not respond to fluid resuscitation. For example, if the patient does not respond to the administration of about 30 ml/kg of intraveneous fluid (or another volume as deemed needed to correct potential intravascular hypovolemia) and the patient has a mean arterial blood pressure below about 65 mmHg.

The selepressin or the composition comprising selepressin may be administered within six hours from when the patient does not respond to fluid resuscitation. For example, the selepressin or composition comprising selepressin may be administered within six hours from when the patient does not respond to the administration of about 30 mL/kg of intravenous fluid (or another volume as deemed needed to correct potential intravascular hypovolemia) and the patient has a mean arterial blood pressure below about 65 mmHg.

The selepressin or the composition comprising selepressin may be administered within six hours from the onset of sepsis.

The patient may have a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may be identified (for example prior to treatment) as having a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may for example be selected for treatment based on having a ratio of partial pressure arterial oxygen to fraction of inspired oxygen (PaO₂/FiO₂) of greater than about 200 prior to treatment, for example from about 200 to about 700 prior to treatment, for example greater than about 260 prior to treatment, for example from about 260 to about 700 prior to treatment, for example greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment.

The selepressin or composition comprising selepressin may be administered to the patient, intravenously, intraperitoneally, intramuscularly, nasally or subcutaneously. Typically, the selepressin or composition comprising selepressin is administered intravenously or subcutaneously. The selepressin or composition comprising selepressin may be administered by infusion, for example continuous infusion, for example an intravenous or subcutaneous infusion, for example intravenous or subcutaneous continuous infusion. Typically, administration is by an intravenous continuous infusion.

The composition comprising selepressin may be in the form of, powders, microparticles, granules, suspensions or solutions, formulated for the intended route of administration.

The composition may comprise for example at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3rd Ed., American Pharmaceutical Association, USA and Pharmaceutical Press UK, 2000.

The composition may be formulated for intravenous administration, for example intravenous continuous infusion. Accordingly, the composition may comprise a sterile aqueous preparation of the selepressin, preferably isotonic with the blood of the recipient. This aqueous preparation may be formulated according to known methods using suitable dispersing or wetting agents and suspending agents. The preparation may also be a sterile injectable solution or suspension in a diluent or solvent, for example a solution in 1,3-butane diol. Other acceptable diluents may include water, Ringer's solution, and isotonic sodium chloride solution. Sterile, fixed oils may be employed as a solvent or suspending medium. Bland fixed oils, including synthetic mono or di-glycerides, and fatty acids, such as oleic acid, may also be used.

The selepressin may be administered in amounts up to about 7.5 ng/kg/min, for example between about 1.25 ng/kg/min and about 7.5 ng/kg/min, for example, between about 1.25 ng/kg/min and about 1.7 ng/kg/min or between about 1.7 ng/kg/min and about 2.5 ng/kg/min, or between about 2.5 and about 3.75 ng/kg/min, or between about 3.5 and about 5.25 ng/kg/min, or between about 5.0 and about 7.5 ng/kg/min.

The selepressin may be administered at an initial infusion dose rate which increases to a maximum infusion dose rate over time. The initial infusion dose rate may be two-thirds of the maximum infusion dose rate. The initial infusion dose rate may be between about 1.7 ng/kg/min and about 5.0 ng/kg/min, for example, about 1.7 ng/kg/min, about 2.5 ng/kg/min, about 3.5 ng/kg/min or about 5 ng/kg/min. The maximum infusion rate (for example, intravenous infusion rate) may be between about 2.5 ng/kg/min and about 7.5 ng/kg/min, for example, about 2.5 ng/kg/min, about 3.75 ng/kg/min, about 5.25 ng/kg/min or about 7.5 ng/kg/min.

The infusion dose rate may start at an initial infusion dose rate of about 1.7 ng/kg/min and increase to a maximum infusion dose rate of about 2.5 ng/kg/min; or the infusion dose rate may start at an initial infusion dose rate of about 2.5 ng/kg/min and increase to a maximum infusion dose rate of about 3.75 ng/kg/min; or the infusion dose rate may start at an initial infusion dose rate of about 3.5 ng/kg/min and increase to a maximum infusion dose rate of about 5.25 ng/kg/min; or the infusion dose rate may start at an initial infusion dose rate of about 5.0 ng/kg/min and increase to a maximum infusion dose rate of about 7.5 ng/kg/min.

The above mentioned dose rates may be for an intravenous or subcutaneous infusion, for example intravenous or subcutaneous continuous infusion.

The treatment duration may be until recovery from the need of vasopressor treatment. If the need of vasopressor treatment subsides and recurs, the treatment can be restarted.

The selepressin or composition comprising selepressin may be administered together with a further antihypotensive agent (vasopressor), for example a catecholamine, for example norepinephrine.

The selepressin or composition comprising selepressin may be administered together with an inotropic agent, for example milrinone dobutamine and/or levosimendan.

According to a further aspect of the invention there is provided selepressin or a composition comprising selepressin for use in treating sepsis in a patient, wherein the patient has a ratio of partial pressure arterial oxygen to fraction of inspired oxygen (PaO₂/FiO₂) of greater than about 260 prior to treatment, for example from about 260 to 700 prior to treatment. There is also provided a method of treating sepsis in a patient, comprising administering selepressin or a composition comprising selepressin to the patient, wherein the patient has a ratio of partial pressure arterial oxygen to fraction of inspired oxygen (PaO₂/FiO₂) of greater than about 260 prior to treatment, for example from about 260 to 700 prior to treatment. There is also provided a use of selepressin or a composition comprising selepressin in the manufacture of a medicament for the treatment of sepsis in a patient, wherein the patient has a ratio of partial pressure arterial oxygen to fraction of inspired oxygen (PaO₂/FiO₂) of greater than about 260 prior to treatment, for example from about 260 to 700 prior to treatment.

Herein, the term “prior to treatment” means prior to administration of the selepressin or the composition comprising selepressin.

For example, the patient may be identified (for example prior to treatment) as having a ratio of partial pressure arterial oxygen to fraction of inspired oxygen (PaO₂/FiO₂) of greater than about 260 prior to treatment. For example, the patient may be selected for treatment based on having a ratio of arterial partial pressure oxygen to fraction of inspired oxygen (PaO₂/FiO₂) of greater than about 260 prior to treatment.

The applicant has found that patients who have a ratio of partial pressure arterial oxygen to fraction of inspired oxygen (PaO₂/FiO₂) greater than about 260 prior to treatment exhibit improved mortality and improved patient outcomes, for example as measured by pressor and mechanical ventilator free days.

The patient may have a ratio of partial pressure arterial oxygen to fraction of inspired oxygen (PaO₂/FiO₂) of greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may be identified (for example prior to treatment) as having a ratio of partial pressure arterial oxygen to fraction of inspired oxygen (PaO₂/FiO₂) of greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment. The patient may for example be selected for treatment based on having a ratio of partial pressure arterial oxygen to fraction of inspired oxygen (PaO₂/FiO₂) of greater than about 270 prior to treatment, for example from about 270 to about 700 prior to treatment, for example greater than about 280 prior to treatment, for example from about 280 to about 700 prior to treatment, for example greater than about 290 prior to treatment, for example from about 290 to about 700 prior to treatment, for example greater than about 300 prior to treatment, for example from about 300 to about 700 prior to treatment, for example from about 300 to about 600 prior to treatment, for example from about 300 to about 500 prior to treatment, for example from about 300 to about 400 prior to treatment.

The sepsis may be vasopressor dependent sepsis or septic shock.

The selepressin or composition comprising selepressin may be administered within six hours from when the patient requires vasopressor therapy.

Patients having sepsis may require vasopressor therapy, for example the administration of a vasopressor, if the patient does not respond to fluid resuscitation. For example, if the patient does not respond to the administration of about 30 ml/kg of intraveneous fluid (or another volume as deemed needed to correct potential intravascular hypovolemia) and the patient has a mean arterial blood pressure below about 65 mmHg.

The selepressin or the composition comprising selepressin may be administered within six hours from when the patient does not respond to fluid resuscitation. For example, the selepressin or the composition comprising selepressin may be administered within six hours from when the patient does not respond to the administration of about 30 mL/of intravenous fluid (or another volume as deemed needed to correct potential intravascular hypovolemia) and the patient has a mean arterial blood pressure below about 65 mmHg.

The selepressin or the composition comprising selepressin may be administered within six hours from the onset of sepsis.

The patient may have a serum lactate concentration of less than about 2 mmol/L prior to treatment. The patient may be identified (for example prior to treatment) as having a serum lactate concentration of less than about 2 mmol/L prior to treatment. The patient may, for example, be selected for treatment based on having a serum lactate concentration of less than about 2 mmol/L prior to treatment.

The selepressin or composition comprising selepressin may be administered to the patient, intravenously, intraperitoneally, intramuscularly, nasally or subcutaneously. Typically, the selepressin or composition comprising selepressin is administered intravenously or subcutaneously. The selepressin or composition comprising selepressin may be administered by infusion, for example continuous infusion, for example an intravenous or subcutaneous infusion, for example intravenous or subcutaneous continuous infusion. Typically, administration is by an intravenous continuous infusion.

The composition comprising selepressin may be in the form of powders, microparticles, granules, suspensions or solutions, formulated for the intended route of administration.

The composition may comprise for example at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3rd Ed., American Pharmaceutical Association, USA and Pharmaceutical Press UK, 2000.

The composition is preferably for intravenous administration, for example, intravenous continuous infusion. Accordingly, the composition may comprise a sterile aqueous preparation of the selepressin, preferably isotonic with the blood of the recipient. This aqueous preparation may be formulated according to known methods using suitable dispersing or wetting agents and suspending agents. The preparation may also be a sterile injectable solution or suspension in a diluent or solvent, for example a solution in 1,3-butane diol. Other acceptable diluents may include water, Ringer's solution, and isotonic sodium chloride solution. Sterile, fixed oils may be employed as a solvent or suspending medium. Bland fixed oils, including synthetic mono or di-glycerides, and fatty acids, such as oleic acid, may also be used.

The selepressin may be administered in amounts up to about 7.5 ng/kg/min, for example between about 1.25 ng/kg/min and about 7.5 ng/kg/min, for example, between about 1.25 ng/kg/min and about 1.7 ng/kg/min or between about 1.7 ng/kg/min and about 2.5 ng/kg/min, or between about 2.5 and about 3.75 ng/kg/min, or between about 3.5 and about 5.25 ng/kg/min, or between about 5.0 and about 7.5 ng/kg/min.

The selepressin may be administered at an initial infusion dose rate which increases to a maximum infusion dose over time. The initial infusion dose rate may be two-thirds of the maximum infusion dose rate. The initial infusion dose rate may be between about 1.7 ng/kg/min and about 5.0 ng/kg/min, for example, about 1.7 ng/kg/min, about 2.5 ng/kg/min, about 3.5 ng/kg/min or about 5 ng/kg/min. The maximum infusion rate may be between about 2.5 ng/kg/min and about 7.5 ng/kg/min, for example, about 2.5 ng/kg/min, about 3.75 ng/kg/min, about 5.25 ng/kg/min or about 7.5 ng/kg/min.

The infusion dose rate may start at an initial infusion dose rate of about 1.7 ng/kg/min and increase to a maximum infusion dose rate of about 2.5 ng/kg/min; or the infusion dose rate may start at an initial infusion dose rate of about 2.5 ng/kg/min and increase to a maximum infusion dose rate of about 3.75 ng/kg/min; or the infusion dose rate may start at an initial infusion dose rate of about 3.5 ng/kg/min and increase to a maximum infusion dose rate of about 5.25 ng/kg/min; or the infusion dose rate may start at an initial infusion dose rate of about 5.0 ng/kg/min and increase to a maximum infusion dose rate of about 7.5 ng/kg/min.

The above mentioned dose rates may be for an intravenous or subcutaneous infusion, for example intravenous or subcutaneous continuous infusion.

The treatment duration may be until recovery from the need of vasopressor treatment. If the need of vasopressor treatment subsides and recurs, the treatment can be restarted.

The selepressin or composition comprising selepressin may be administered together with a further antihypotensive agent (vasopressor), for example a catecholamine, for example norepinephrine.

The selepressin or composition comprising selepressin may be administered together with an inotropic agent, for example milrinone, dobutamine, and/or levosimendan.

Selepressin can be synthesized by methods known in the art, for example as set out in WO2006020491, for example from page 9 to 14, the contents of which is incorporated herein by reference.

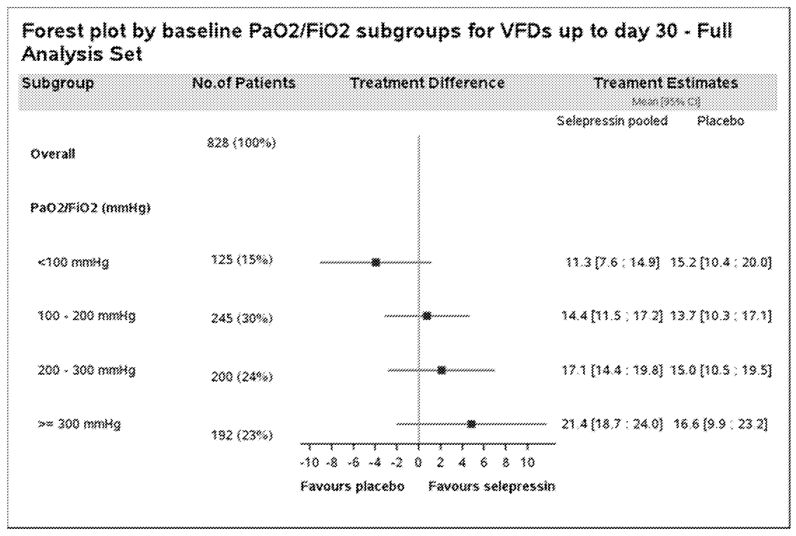

Specific embodiments of the invention will now be described, by way of example only, with reference to the following examples and FIG. 1.

### EXAMPLES

### Example 1

### General Description of In Vivo Studies

The study described in the following examples was a randomized, placebo-controlled, seamless two-part, adaptive clinical study. The primary objective of the study was to demonstrate the superiority of selepressin plus standard care versus placebo plus standard care in the number of vasopressor- and mechanical ventilator-free days (with penalty for mortality) in patients with vasopressor-dependent sepsis and septic shock. The term “vasopressor-dependent sepsis” includes all patients with sepsis requiring vasopressor therapy. Septic shock includes patients with sepsis who require vasopressor therapy and who have a serum lactate concentration of ≥2 mmol/L.

The inclusion and exclusion criteria for the trial are set out below.

Inclusion Criteria
18 years of age or older
Proven or suspected infection Septic shock defined as hypotension (systolic blood pressure less than 90 mm Hg or
Mean arterial pressure less than 65 mm Hg requiring vasopressor treatment (i.e., any dose of norepinephrine/noradrenaline base greater than 5 mg/min) despite adequate fluid resuscitation (at least 1 litre for hypotension)
Informed consent obtained in accordance with local regulations

Exclusion Criteria
Unable to initiate treatment with selepressin or placebo within 12 h of onset of vasopressor treatment for septic shock
Primary cause of hypotension not due to sepsis
Previous severe sepsis with intensive care admission within this hospital stay
Known/suspected acute mesenteric ischemia
Suspicion of concomitant acute coronary syndrome based on clinical symptoms and/or electrocardiogram findings during this episode of septic shock
Chronic mechanical ventilation for any reason or severe chronic obstructive pulmonary disease (COPD) requiring either continuous daily oxygen use during the preceding 30 days or mechanical ventilation (for acute exacerbation of COPD) during the preceding 30 days
Received bone marrow transplant during the preceding 6 months or chemotherapy during the preceding 30 days for lymphoma or leukemia
Known to be pregnant Decision to limit full care taken before obtaining informed consent
Use of vasopressin in the past 12 hours before start of selepressin or placebo or use of terlipressin within 7 days of start of selepressin or placebo
Prior enrollment in the study
Prior use of an investigational medicinal product within the last month or planned or concurrent participation in a clinical trial for any investigational drug or investigational device

Additional Criteria that Must be Met at the Time of the Start of the Infusion of Selepressin or Placebo
Received a minimum of 30 ml/kg fluid in total from the onset of hypotension (not required if there is evidence of adequate fluid repletion or overload)
Received a continuous infusion of norepinephrine/noradrenaline base greater than 5 mg/min for at least 1 h and is still receiving at least 5 mg/min norepinephrine/noradrenaline base
Less than 12 h since onset of vasopressor treatment for septic shock

The targeted study population is a typical sample of patients presenting with vasopressor-dependent sepsis, defined by failing to respond to intravenous fluid replacement and requiring vasopressors for at least 1 hour, in whom treatment with selepressin or placebo could be initiated within 12 hours of the initiation of vasopressors.

The study drug was the continuous intravenous infusion of either the experimental agent or a matching placebo. In all instances, subjects received study infusion and standard care. The possible treatment arms were: Placebo; selepressin starting infusion rate 1.7 ng/kg/min to maximum 2.5 ng/kg/min; selepressin starting 2.5 ng/kg/min to maximum 3.75 ng/kg/min; selepressin starting 3.5 ng/kg/min to maximum 5.25 ng/kg/min; and selepressin starting 5.0 ng/kg/min to 7.5 ng/kg/min. All arms had an initial infusion rate that is two-thirds of the maximum, with titration of study drug guided by a detailed administration guide that ensured titration to specific blood pressure targets. This latter approach sought to wean all other vasoactive agents as soon as possible, and in a specified order, beginning with any open-label vasopressor other than norepinephrine, followed by norepinephrine, and finally the blinded study infusion. All patients received the study infusion until recovery from the need of vasopressor treatment or for 30 days, whichever came first. If the need of vasopressor treatment subsided and recurred within the 30-day treatment period, the initially allocated treatment was restarted.

The primary endpoint for the trial was the number of pressor- and mechanical ventilator-free days (P&VFDs) up to Day 30. This composite endpoint was defined as the number of days (recorded in tenths) from the start of treatment with selepressin or placebo to 30 days thereafter, during which the patient was alive, free of treatment with intravenous vasopressors including the study drug, and free of any invasive mechanical ventilation. Any patient who died within this 30-day period was assigned zero P&VFDs, even if there was a period during which the patient was free of both vasopressor treatment and mechanical ventilation. If vasopressors needed to be restarted or mechanical ventilation needed to be initiated or restarted, and the use of either was greater than 60 minutes within a 24-hour period, then the clock was reset to zero and the patient was not considered free of vasopressors and mechanical ventilation until after those therapies were again discontinued. Vasopressor use or mechanical ventilation during and up to 3 hours after a surgery or procedure (including bedside procedures) was exempt from this rule and did not reset the calculation of P&VFDs.

For the purpose of defining P&VFDs, vasopressor use was defined as any intravenous dose of norepinephrine, phenylephrine, dopamine, epinephrine, vasopressin, and the study drug (i.e., selepressin or placebo). Mechanical ventilation was defined as the use of endotracheal or tracheostomy tube-assisted ventilation (greater than 5 cm H₂O continuous positive airway pressure and greater than 5 cm H₂O of pressure support from the ventilator in patients with tracheostomy).

Subsequent analysis of the data generated in the study of Example 1 has led to the following findings described in Examples 2 to 5.

### Example 2. Effect of Treatment with Selepressin within Six Hours from when the Patient Required the Administration of a Vasopressor

The effect of initiation of selepressin treatment from zero up to six hours compared with initiation of selepressin treatment at six hours or later was evaluated. Outcomes for all patients treated with selepressin at any dose were pooled and compared with placebo-treated patients in the same time treatment cohort. Compared with placebo, selepressin treatment between zero and up to six hours from when the patient required the administration of a vasopressor resulted in an improvement in estimated P&VFDs (2.08 more) and mortality (6.57% less); whereas selepressin treatment at six hours or later led to similar outcomes as placebo treatment, as set out in Table 1 below

TABLE 1

| | <6 hours | | | ≥6 hours | | |
|---|---|---|---|---|---|---|
| | Placebo | Selepressin | Treatment | Placebo | Selepressin | Treatment |
| | (n = 79) | (n = 147) | difference | (n = 187) | (n = 415) | difference |
| Estimated | 12.49 | 14.56 | 2.08 | 15.64 | 15.59 | −0.05 |
| Pressor and | | | | | | |
| Ventilator | | | | | | |
| Free days | | | | | | |
| (P&VFDs): | | | | | | |
| all patients | | | | | | |
| Estimated | 42.30% | 35.73% | −6.57% | 30.88% | 33.79% | 2.91% |
| 30- day | | | | | | |
| mortality | | | | | | |
| (up to day | | | | | | |
| 30) | | | | | | |
| Estimated | 21.64 | 22.66 | 1.02 | 22.63 | 23.55 | 0.92 |
| Pressor and | | | | | | |
| Ventilated | | | | | | |
| Free Days | | | | | | |
| (P&VFDs): | | | | | | |
| survivors | | | | | | |
| only | | | | | | |

### Example 3. Effect of Treatment with Selepressin in Patients Stratified by Serum Lactate at Baseline

The outcomes in patients treated with selepressin who had serum lactate <2 mmol/L at baseline, that is prior to administration of the selepressin, were compared with outcomes in patients who had serum lactate ≥2 mmol/L at baseline, relative to the placebo-treated patients in the respective lactate-stratified cohorts. Outcomes for all patients treated with selepressin at any dose were pooled and compared with placebo-treated patients in the same lactate cohort. Compared with placebo, selepressin treatment in patients who had serum lactate <2 mmol/L at baseline resulted in an improvement in estimated P&VFDs (2.34 more) and mortality (6.32% less) compared with placebo; whereas selepressin treatment in patients who had serum lactate ≥2 mmol/L at baseline led to similar outcomes as placebo treatment, as shown in Table 2.

TABLE 2

| | Lactate <2 mmol/L | | | Lactate ≥2 mmol/L | | |
|---|---|---|---|---|---|---|
| | Placebo | Selepressin | Treatment | Placebo | Selepressin | Treatment |
| | (n = 82) | (n = 184) | difference | (n = 173) | (n = 340) | difference |
| Est | 15.19 | 17.53 | 2.34 | 14.54 | 13.87 | −0.67 |
| P&VFDs: | | | | | | |
| all patients | | | | | | |
| Est % | 27.71% | 21.39% | −6.32% | 36.09% | 40.04% | 3.96% |
| mortality | | | | | | |
| Est | 21.01 | 22.30 | 1.29 | 22.75 | 23.14 | 0.38 |
| P&VFDs: | | | | | | |
| survivors | | | | | | |

### Example 4. Effect of Treatment with Selepressin in Patients Stratified by PaO2/FiO2 at Baseline

The outcomes in patients treated with selepressin who had PaO2/FiO2≥200 mmHg at baseline were compared with outcomes in patients who had PaO2/FiO2<200 mmHg at baseline, relative to the placebo-treated patients in the respective PaO2/FiO2-stratified cohorts. Outcomes for all patients treated with selepressin at any dose were pooled and compared with placebo-treated patients in the same PaO2/FiO2 cohort. As illustrated in FIG. 1, compared with placebo, selepressin treatment in patients who had PaO2/FiO2≥200 mmHg at baseline resulted in an improvement in estimated VFDs (2.1 more for PaO2/FiO2 200-300 mmHg; 4.8 more for ≥300 mmHg) compared with placebo; whereas selepressin treatment in patients who had PaO2/FiO2<200 mmHg at baseline led to similar outcomes as placebo treatment (100-200 mmHg) or slightly worse (<100 mmHg).

### REFERENCES

1. Russell, James A. et al., V1A agonist is an effective substitute for norepinephrine in phase IIa randomized, placebo-controlled trial in septic shock patients, Critical Care, 2017, 21(213), 1-10
2. Russell, James A. et al., Vasopressin versus Norepinephrine Infusion in Patients with Septic Shock, N Engl J Med, 2008, 358(9), 877-887.
3. Lewis, Rojer J., Rationale and Design of an Adaptive Phase 2b/3 Clinical Trial of Selepressin for Adults in Septic Shock, Annals ATS, 2018, 15(2), 250-257

What is claimed is:

1. A composition comprising selepressin for use in treating sepsis in a patient requiring vasopressor therapy, wherein the composition comprising selepressin is administered to the patient within six hours from when the patient requires the vasopressor therapy.

2. A composition comprising selepressin for use according to claim 1, wherein the patient has, or is identified as having, a ratio of partial pressure arterial oxygen to fraction of inspired oxygen (PaO₂/FiO₂) of greater than about 200 prior to treatment.

3. A composition comprising selepressin for use according to claim 1 or 2, wherein the patient has, or is identified as having, a serum lactate concentration of less than about 2 mmol/L prior to treatment.

4. A composition comprising selepressin for use in treating sepsis in a patient, wherein the patient has a serum lactate concentration of less than 2 mmol/L prior to treatment.

5. A composition for use according to claim 4, wherein the patient is identified as having a serum lactate concentration of less than about 2 mmol/L prior to treatment.

6. A composition comprising selepressin for use in treating sepsis in a patient, wherein the patient has a ratio of partial pressure arterial oxygen to fraction of inspired oxygen (PaO₂/FiO₂) of greater than about 260 prior to treatment.

7. A composition for use according to claim 6, wherein the patient is identified as having a ratio of partial pressure arterial oxygen to fraction of inspired oxygen (PaO₂/FiO₂) of greater than about 260 prior to treatment.

8. A composition comprising selepressin for use according to claim 6 or 7, wherein the ratio of partial pressure arterial oxygen to fraction of inspired oxygen (PaO₂/FiO₂) is greater than about 300 prior to treatment.

9. A composition comprising selepressin for use according to any preceding claim, wherein the sepsis is vasopressor dependent sepsis.

10. A composition comprising selepressin for use according to any one of claim 1, 2, 6, 7 or 8, wherein the sepsis is septic shock.

11. A method of treating sepsis in a patient requiring vasopressor therapy, comprising administering a composition comprising selepressin to the patient within six hours from when the patient requires the vasopressor therapy.

12. A method according to claim 11, wherein the patient has, or is identified as having, a serum lactate concentration of less than about 2 mmol/L prior to treatment.

13. A method according to any one of claim 11 or 12, wherein the patient has, or is identified as having, a ratio of partial pressure arterial oxygen to fraction of inspired oxygen (PaO₂/FiO₂) of greater than about 200 prior to treatment.

14. A method of treating sepsis, comprising administering a composition comprising selepressin to a patient in need thereof, wherein the patient has a serum lactate concentration of less than 2 mmol/L prior to treatment.

15. A method according to claim 14, wherein the patient is identified as having a serum lactate concentration of less than about 2 mmol/L prior to treatment.

16. A method of treating sepsis, comprising administering a composition comprising selepressin to a patient in need thereof, wherein the patient has a ratio of partial pressure arterial oxygen to fraction of inspired oxygen (PaO₂/FiO₂) of greater than about 260 prior to treatment.

17. A method according to claim 16, wherein the patient is identified as having a ratio of partial pressure arterial oxygen to fraction of inspired oxygen (PaO₂/FiO₂) of greater than about 260 prior to treatment.

18. A method according to claim 16 or 17, wherein the ratio of partial pressure arterial oxygen to fraction of inspired oxygen (PaO₂/FiO₂) is greater than about 300 prior to treatment.

19. A method according to any one of claims 11-18, wherein the sepsis is vasopressor dependent sepsis.

20. A method according to any one of claim 11, 13, 16, 17 or 18, wherein the sepsis is septic shock.

21. A composition comprising selepressin for use or method according to any preceding claim, wherein the selepressin is administered intravenously or subcutaneously.

22. A composition comprising selepressin for use or method according to any preceding claim, wherein the selepressin is administered by continuous intravenous infusion at a dose rate from about 1.7 ng/kg/min to about 7.5 ng/kg/min.

23. A composition comprising selepressin for use or method according to claim 22, wherein the selepressin is administered by continuous intravenous infusion at a starting intravenous infusion dose rate between about 1.7 ng/kg/min and about 5.0 ng/kg/min; and a maximum intravenous infusion dose rate between about 2.5 ng/kg/min and about 7.5 ng/kg/min.

24. A composition comprising selepressin for use or method according to any preceding claim, wherein the selepressin is administered together with a further antihypotensive agent.

25. A composition comprising selepressin for use or method according to any preceding claim, wherein the selepressin is administered together with an inotropic agent.
